(19) United States
(12) Patent Application Publication
(10) Pub. No.: US 2017/0096486 A1
(43) Pub. Date: Apr. 6, 2017

(54) AGENTS INHIBITING GRANULIN FOR TREATMENT OF CANCER

(71) Applicant: GORAN LANDBERG, Gothenburg (SE)

(72) Inventor: GORAN LANDBERG, Gothenburg (SE)

(21) Appl. No.: 15/128,519

(22) PCT Filed: Mar. 26, 2015
(86) PCT No.: PCT/EP2015/056663

§ 371 Date: Sep. 23, 2016

(30) Foreign Application Priority Data
Mar. 26, 2014 (GB) 1405449.8
Oct. 20, 2014 (GB) 1418640.7

(51) Int. Cl. C07K 16/28; G01N 33/50; A61K 31/7088; A61K 45/06; A61K 39/395; C12N 15/113

(52) U.S. Cl. CPC C07K 16/286; A61K 39/39558; C12N 15/113; A61K 31/7088; A61K 45/06; G01N 33/5011; C07K 2317/76; C12N 2310/11; C12N 2310/14; A61K 2039/505

(57) ABSTRACT

The disclosure provides agents that inhibit granulin signalling for use as medicaments to reduce cancer stem cell activity in the treatment of cancer. The disclosure also provides agents that inhibit soluble granulin signalling for use in the treatment of cancer. The treatments of the disclosure may be of particular utility in breast cancer; prostate cancer; and melanoma, and are also of use in treatment of cancers associated with hypoxic tumours. Suitable agents may include those that inhibit granulin expression, inhibit granulin cleavage, or bind to and inhibit soluble granulin. Such agents may be used in combination with inhibitors of angiogenesis.

The present invention relates to methods of reducing cancer stem cell activity, the medical use of agents to reduce cancer stem cell activity in the treatment of cancer, and medical uses of agents in the treatment of cancer.

The invention also relates to methods of reducing cancer stem cell activity in a subject, and to methods of treating cancer in a subject.

The invention further relates to methods by which agents may be screened for their suitability for use in methods of reducing cancer stem cell activity, medical use to reduce cancer stem cell activity in the treatment of cancer, and medical use in the treatment of cancer.

The invention also relates to cell culture media and supplements.

### BACKGROUND

Treatment of solid tumours such as breast cancer often target the bulk of the tumour population leaving behind treatment resistant cells with stem cell like properties. These stem cell-like cells have been implicated in the generation of recurrent disease (Rich J N and Bao S. Chemotherapy and cancer stem cells. Cell Stem Cell 2007, Oct.; 11 1(4): 353-355; and Pajonk F, Vlashi E, McBride W H. Radiation resistance of cancer stem cells: The 4 R's of radiobiology revisited. Stem Cells 2010, April; 28(4): 639-648). The factors that influence the numerical population of breast cancer stem cells (BCSC) are therefore of huge interest. Recently it has become evident that microenvironmental factors contribute to influencing progenitor cell de-differentiation to stem cell-like phenotypes. Solid tumours create a complex microenvironment, consisting of a mixture of inflammatory cells as well as a heterogeneous tumour population. This complex cell population results in constant cell-cell interactions which may regulate stem cell populations in a solid tumour. Another, common environmental niche in solid tumours is associated with the development of hypoxia. This occurs when oxygen levels decrease within a tumour due to a de-vasularisation process as the tumour becomes large in size. The expression of HIF1α (Hypoxia inducible factor alpha) has been observed to drive stem cell like phenotype in many carcinomas including breast. Although the influence of the microenvironment of tumours has on the cancer cells has been studied in detail, the cancer stem cells response and influence on the microenvironment has not been fully clarified.

Progranulin (PGRN), also known as PC cell-derived growth factor (PCDGF), or granulin/epithelin precursor, is an 88-kDa glycoprotein (GP88) composed of 7.5 cysteine-rich tandem repeats. PRGN is characterized as an autocrine growth factor which stimulates important tumorigenesis steps, including proliferation, estrogen-independence, survival, migration, invasion and angiogenesis. PRGN is also a poor prognostic factor as it inhibits tamoxifen-induced apoptosis and alters the cell growth response to estrogen and tamoxifen in vivo. This peptide has been implicated in tamoxifen resistance by multiple mechanisms including estrogen-independent tumor proliferation, inhibition of tamoxifen-induced poly ADP-ribose polymerase (PARP) cleavage, and inhibition of apoptosis by down-regulating tamoxifen-induced bcl-2 or promotion of tumor angiogenesis.

In breast cancer, progranulin expression has been observed to be estrogen regulated and highly expressed compared to normal breast tissue (Lu and Serrero, 1999). The association of PRGN gene expression with breast cancer was confirmed when the transcriptome profiles of normal and transformed mammary epithelia were compared (Leerkes et al., 2002). Using shotgun sequencing of open reading frame expressed sequence tags (ORESTES), global gene expression from a pool of 24 breast cancer cell lines was compared with that of purified normal mammary cells (Leerkes et al., 2002). The PRGN gene was detected with an incidence of 17:1 between tumor and normal breast epithelia, (from 37,980 tumor and 21,437 normal sequences).

### SUMMARY OF THE INVENTION

In a first aspect, the invention provides the use of an agent that inhibits granulin signalling to reduce cancer stem cell activity.

In a second aspect, the invention provides an agent that inhibits granulin signalling for use as a medicament to reduce cancer stem cell activity in the treatment of cancer.

In a third aspect, the invention provides an agent that inhibits granulin signalling for use as a medicament in the treatment of cancer.

In a fourth aspect, the invention provides a method of reducing cancer stem cell activity in a subject, the method comprising providing to a subject in need of such a reduction in cancer stem cell activity an amount of an agent that inhibits granulin signalling sufficient to reduce cancer stem cell activity.

In a fifth aspect, the invention provides a method of reducing cancer stem cell activity in a subject, the method comprising providing to a subject in need of such a reduction in cancer stem cell activity an amount of an agent that inhibits sortilin activity sufficient to reduce cancer stem cell activity.

In a sixth aspect, the invention provides a method of screening an agent for suitability for use in methods of reducing cancer stem cell activity, medical use to reduce cancer stem cell activity in the treatment of cancer, and medical use in the treatment of cancer, the method comprising assessing the ability of the agent to inhibit granulin signalling

In a seventh aspect, the invention provides a cell culture product comprising granulin or a precursor or biologically active fragment of granulin.

In an eighth aspect the invention provides a method of promoting survival in cultured stem cells, the method comprising providing the cultured stem cells with a source of granulin, or a precursor or biologically active fragment of granulin.

In a ninth aspect the invention provides an agent that inhibits sortilin activity for use as a medicament to reduce cancer stem cell activity in the treatment of cancer.

In a tenth aspect the invention provides an agent that inhibits sortilin activity for use as a medicament in the treatment of cancer.

In an eleventh aspect the invention provides a method of screening an agent for suitability for use in methods of reducing cancer stem cell activity, medical use to reduce cancer stem cell activity in the treatment of cancer, and medical use in the treatment of cancer, the method comprising assessing the ability of the agent to inhibit sortilin activity.

In a twelfth aspect, the invention provides the use of an agent that inhibits sortilin activity to reduce cancer stem cell activity.

The inventors believe the methods of the first aspect of the invention, and the agents for medical use of the second and third aspects of the invention, to be applicable to a wide range of cancers, as discussed further below. Suitably, the cancer may be breast cancer, and particularly suitably the cancer may be luminal A breast cancer.

In the present disclosure, references to “uses of the invention” should, except for where the context requires otherwise, be taken as encompassing the use set out in the first aspect of the invention, as well as the medical uses set out in the second and third aspects of the invention.

Similarly, references to “methods of the invention” should, except for where the context requires otherwise, be taken as encompassing any of the methods of treatment of the fourth or fifth aspects of the invention, the method of screening of the sixth aspect of the invention, or the method of promoting survival in cultured stem cells of the eighth aspect of the invention.

References to “agents of the invention” should be taken as encompassing agents that inhibit granulin signalling when employed in the uses of the invention, or in the methods of the invention.

### DETAILED DESCRIPTION OF THE INVENTION

To improve the understanding of the invention, useful definitions will now be provided in respect of certain terms used in the present disclosure.

### “Granulin Signalling”

For the purposes of the present disclosure references to “granulin signalling” should be construed as encompassing signalling by granulin and its precursors (such as progranulin), and products of the cleavage of progranulin or granulin (including paragranulin and the granulin 1 or 5 domains), such as by metalloproteases.

“Agents that Inhibit Granulin Signalling”

In their broadest embodiments, the methods or uses of the invention may be put into practice using any agent that inhibits granulin signalling. It will be appreciated that suitable agents may achieve this required inhibition in granulin signalling by a number of different approaches. Suitably, an agent that inhibits granulin signalling may be one that reduces granulin expression, thus decreasing the amount of granulin that is available to take part in signalling. A suitable agent that inhibits granulin signalling may be one that reduces the amount of a form of granulin associated with signalling that is produced, for example, by reducing the conversion of granulin, or an associated molecule, into a soluble form. A still further suitable form of an agent that inhibits granulin signalling may one that binds to granulin and thereby inhibits its ability to generate a signal. Another embodiment of an agent that inhibits granulin signalling may be one that interferes with the ability of a receptor to bind granulin, thereby reducing the extent of signal generation.

Without detracting from the broad embodiments set out above, certain particular agents that inhibit granulin signalling, or groups of such agents, are particularly suitable for use in specific aspects of the methods or uses of the invention. Guidance regarding some of these is provided in the following paragraphs.

In a suitable embodiment, the agent that inhibits granulin signalling is one that inhibits soluble granulin signalling. The inventors believe that soluble granulin signalling plays a major role in promoting cancer stem cell activity. This is supported by results reported in the Experimental Results section below, which show that the cancer stem cell promoting activities are able to be transferred via conditioned media.

Granulin signalling in the local cancer milieu may contribute to the promotion of cancer stem cell activity. The influence of paracrine granulin signalling on cancer stem cell activity has not previously been recognised, but this finding gives rise to beneficial embodiments of the present invention. Accordingly, a suitable agent for use in the methods or uses of the invention may be one that inhibits paracrine granulin signalling. Such an agent may be provided in an amount sufficient to inhibit paracrine granulin signalling.

Alternatively, or additionally, a suitable agent may be one that inhibits endocrine granulin signalling.

As referred to above, a suitable agent for use in the methods or uses of the invention may be an inhibitor of granulin cleavage. In such embodiments, a therapeutically effective amount of the agent may be an amount sufficient to prevent generation of soluble granulin fragments.

In a suitable embodiment, an inhibitor of granulin cleavage and/or progranulin cleavage suitable for use as an agent of the invention is selected from the group consisting of: an inhibitor of elastase activity; an inhibitor of proteinase 3 activity; an inhibitor of cathepsin G activity; an inhibitor of MMP-12 activity; and an inhibitor of MMP-9 activity. All of these enzymes have the capacity to cleave granulin, yielding soluble granulin and other cleavage products that can cause paracrine or endocrine granulin signalling. Accordingly, inhibitors of these enzymes are able to usefully inhibit this generation and thus inhibit non-juxtacrine granulin signalling.

Products with granulin signalling activity are particularly generated on the cleavage of granulin by elastase. Thus inhibitors of elastase activity are highly suitable for use as agents that inhibit granulin signalling (and particularly non-juxtacrine granulin signalling, such as paracrine or endocrine signalling).

A suitable agent that inhibits elastase activity may be selected from the group consisting of: α1-antitrypsin; elastase inhibitor I; elastase inhibitor II; elastase inhibitor III; N-methoxysuccinyl-Ala-Ala-ProVal-chloromethyl ketone; and secreted leukocyte protease inhibitor (SPLI).

In another suitable embodiment, an agent for use in the methods or uses of the invention may be one that binds to and neutralises granulin. Suitably such an agent may be one that binds to and neutralises soluble granulin.

Merely by way of example, an agent suitable for use in such embodiments may be selected from the group consisting of: an anti-granulin antibody; a small molecule inhibitor of extracellular granulin; and a mimic of known natural binding partners of granulin, such as a soluble granulin receptor, or a mimetic of the tumour necrosis factor receptor (TNFR) or sortilin.

Suitably the agent that inhibits granulin signalling may be an agent that inhibits progranulin signalling via sortilin. Suitably the agent may be one that inhibits binding of progranulin to sortilin. In a suitable embodiment an agent of this sort may bind to sortilin in a manner that prevents the binding of progranulin to this receptor.

Alternatively, a suitable agent may be a soluble fragment of sortilin, or a variant thereof. Agents in accordance with this embodiment are able to bind progranulin in the extracellular milieu, thereby preventing the binding of progranulin to cellular sortilin. Thus such agents are able to inhibit granulin signalling.

Merely by way of example, suitable polyclonal antibodies that may be used as agents of the invention include the anti-progranulin antibody available from R&D Systems under the catalogue number #af2420; and the anti-progranulin antibody available from Life Technologies under the catalogue number #40-3400.

The skilled person will appreciate that binding fragments of anti-granulin antibodies may be used as an alternative to full length antibodies themselves.

As referred to above, in a suitable embodiment an agent that inhibits granulin signalling may be one that interferes with the ability of a receptor to bind granulin, thereby reducing the extent of signal generation. Merely by way of example, such agents may include antibodies, or other such molecules, that bind to the receptor in a manner that prevents access of granulin to its binding site, and thus prevent effective (which is to say, signal-inducing) binding by granulin.

It will be appreciated that the methods or uses of the invention may make use of combinations of different types of agents that inhibit granulin signalling.

In order to achieve their aims, agents that inhibit granulin signalling should be provided in therapeutically effective amounts when practicing the methods or uses of the invention. It will be recognised that a suitable therapeutic amount may be provided by means of a single administration, or may be provided over the course of a number of administrations. Medicaments or pharmaceutical compositions may be formulated appropriately with this in mind to provide such therapeutically effective amounts.

“Agents that Inhibit Sortilin Activity”

The recognition that cancer stem cell activity can be mediated by granulin signalling via the sortilin receptor also gives rise to the fifth, ninth, tenth, eleventh, and twelfth aspects of the invention.

The skilled person will be able to identify a number of different agents that are capable of inhibiting sortilin activity. Merely by way of example, these may include agents that inhibit the binding of granulin or progranulin to sortilin (such as function-blocking anti-sortilin antibodies), agents that inhibit the activity of sortilin on binding of molecules such as granulin or progranulin, and agents that reduce sortilin expression (such as siRNA, RNAi, antisense oligonucleotides, or other “gene silencing” techniques).

Except for where the context requires otherwise, considerations in the present document regarding factors such as the clinical contexts in which agents may be used, cancers to be treated, formulations, and the like set out in respect of agents that inhibit granulin signalling should also be taken as applying to agents that inhibit sortilin activity.

### “Cancer Stem Cell Activity”

As set out above, the inventors have found that agents that inhibit granulin signalling are able to reduce cancer stem cell activity. For the purposes of the present disclosure cancer stem cell activity may be taken as the ability for a single cancer cell to self-renew, and this can be measured by the ability of cells to form mammospheres. Hence a reduction in cancer stem cell activity may be determined by determining whether a reduction in mammosphere formation has occurred.

Cancer stem cell activity may be demonstrated by the capacity of cells to form mammospheres during culture in vitro. It will therefore be appreciated that a reduction in cancer stem cell activity may be demonstrated by a reduction in the propensity of cells to form mammospheres during culture in vitro. Suitable assays by which mammosphere formation may be investigated and quantified, thus allowing a determination to be made as to whether or not cancer stem cell activity has been reduced, are described further in the Experimental Results section.

An alternative method by which cancer stem cell activity may be demonstrated is the ability of putative cancer stem cells to initiate tumour formation when introduced to a host, such as an immunocompromised mouse. In these methods, a reduction in cancer stem cell activity may be demonstrated by a reduction in the propensity of cells to form tumours in the host. Suitable examples of assays of this sort are also described further in the Experimental Results section.

Assays such as those referred to above may be used to assess the suitability of a putative agent believed to inhibit granulin signalling for use in the methods or uses of the invention. They may also be used to identify suitable therapeutically effective doses of agents that inhibit granulin signalling activity.

As referred to above, cancer stem cells exhibit increased resistance to treatment, whether by chemotherapy or radiotherapy, and the activity of these cells is considered to contribute to the development of resistant cancers. Thus the uses, methods and agents of the invention are particularly applicable to the treatment of resistant cancers that do not respond to other treatments.

### “Treatment of Cancer”

In the context of the present invention, “treatment” of cancer should be taken as encompassing not only the treatment of existing cancer, but also the prevention of development of cancer. Further discussion of these embodiments is provided below.

Preventative treatment of cancer with the methods of uses of the invention may be of particular benefit in subjects identified as having an elevated risk of developing cancer. Such an elevated risk may be caused by genetic predisposition to cancer formation. A predisposition of this sort may be determined by genetic testing, or by familial history of incidences of cancer.

As has been referred to above, “treatment” of cancer in the context of the present invention encompasses the treatment of existing cancers at various clinical stages. Certain of these uses are particularly indicated by the inventors' novel finding that granulin signalling is associated with the regulation of cancer stem cell activity. Without this knowledge, which is disclosed for the first time in the present application, the skilled person would not have believed that inhibitors of granulin signalling would have been therapeutically effective in a number of these contexts.

One application in which the methods and uses of the invention may be of particular use is as adjuvant therapy. Accordingly, this represents a suitable embodiment of the invention. Adjuvant therapies are used after primary treatment, such as surgery (for example after mastectomy or lumpectomy in the case of breast cancer) to reduce the ability of any remaining cancer stem cells to initiate or otherwise contribute to the reconstitution of a tumour. The problem of remaining cancer stem cells is one that is well recognised by those practicing in this area of medicine (see, for example, the articles by Rich et al. or Pajonk et al. referred to in the “Background” above). Consequently, current surgical approaches go to great lengths to prevent the continued presence of any cancerous tissue, which may contain cancer stem cells able to reconstitute the tumour. Indeed, surgery normally involves the removal of surrounding non-cancerous (normal) tissue, specifically to ensure that no cells able to reconstitute the cancer remain.

It will be appreciated that the ability of the methods and uses of the invention to inhibit cancer stem cell activity are of great utility in clinical contexts of the sort described above. The ability to use agents that inhibit granulin signalling to reduce the activity of cancer stem cells provides treatments that will allow clinicians greater confidence that cancers that have already undergone treatment will not return. This is expected to be applicable across a range of cancer treatment types, including surgical treatment, chemotherapy or radiotherapy. Even in the event that some cancer stem cells remain after the initial anti-cancer therapy, treatment using the methods or uses of the invention will reduce the activity of cancer stem cells, and inhibit their ability to contribute to the formation of a new tumour.

It is anticipated that the treatments with the methods or uses of the present invention will allow a reduction in the extent of anti-cancer treatments that need be applied, since the need to “over treat” in order to avoid the risk of cancer stem cells remaining will be reduced. This may help to reduce the adverse side effects experienced by patients undergoing chemotherapy or radiotherapy for treatment of cancer, and also reduce the extent to which it is necessary to excise beyond the area believed to be cancerous during surgical treatment.

Recent research has also drawn attention to the importance of cancer stem cells in the propagation and development of metastatic cancers. The ability of the methods and uses of the invention to reduce cancer stem cell activity thus makes them very well suited to use in the treatment of metastatic cancer. This activity also makes the methods and uses of the invention well suited to use in the prevention of metastases.

Generally, when used in the treatment of metastatic cancer, the methods or uses of the invention may be put into practice as soon as early as possible once the presence of metastases has been identified.

Alternatively, when used for prevention, the methods or uses of the invention may be employed prophylactically, when a risk of metastasis has been identified, but before the metastatic process has begun.

Metastasising cancers may cause the initiation of new tumours at sites that are quite distant from the primary tumour, and the location of such metastases may be difficult to predict. Systemic treatment using agents that inhibit granulin signalling in the methods or uses of the invention may thus be of benefit in the treatment or prevention of metastases, since these are able to reduce cancer stem cell activity at any location where this is required, without the need to identify, and attempt to target, specific sites where metastases may be forming.

Not detracting from the above, and without wishing to be limited by any hypothesis, the inventors believe that the methods and uses of the invention may be of benefit in a range of different contexts in vivo where, unless inhibited, granulin signalling otherwise promotes cancer stem cell activity. Merely by way of example, granulin activity is elevated in response to various cues known to be associated with cancer, including hypoxia and inflammation.

Hypoxia occurs naturally in a number of different situations associated with various cancer forms. For example, early stages of cancer formation, such as at metastases, may be hypoxic since cancer cells are proliferating without adequate blood vessel formation having had an opportunity to occur. The benefits of the methods and uses of the invention in the prevention and/or treatment of metastases have already been discussed above, but this ability to inhibit, and thereby counteract, hypoxia-induced increases in granulin expression and signalling identifies a route by which such beneficial effects may be achieved.

Hypoxia is also associated with well established solid tumours. In these cases, the density of cells compared to the available blood supply may be such that hypoxic niches develop. Granulin signalling in these may contribute to a milieu that promotes cancer stem cell activity. The use of agents that inhibit granulin signalling is able to counteract these conditions. Thus the methods and uses of the invention may be of great utility in the treatment of solid tumours, particularly those in which hypoxia is established.

In addition to these naturally occurring contexts discussed above, hypoxia may also arise in response to cancer treatment. For example, certain anti-cancer agents achieve their effect through the inhibition of angiogenesis, in an attempt to limit the availability of oxygen and nutrients to the cancer cells, and thus limit their proliferation. Furthermore the radiation treatment used during radiotherapy for cancer can induce hypoxia.

Accordingly, as discussed elsewhere in the present disclosure, the methods and uses of the invention may advantageously be provided in combination with other anti-cancer treatments that give rise to hypoxia, in order to avoid the tendency of this induced hypoxia to promote cancer stem cell activity.

It is also worth noting that in certain circumstances hypoxia-inducible transcription factors (HIFs) are activated even without a corresponding reduction in availability of oxygen in the cellular environment. Merely by way of example, HIF-1 alpha expression can be induced by growth factor signalling to mimic hypoxia. Growth factors able to induce the expression of HIF-1 alpha in this manner include heregulin, epidermal growth factor (EGF), insulin growth factor (IGF) and transforming growth factor (TGF). The methods or uses of the invention may be of use in the prevention and/or treatment of cancers where growth factor signalling of this sort is associated with cancer stem cell activity.

Granulin expression is also found associated with inflammation. Inflammation is known to be associated with the development and progression of certain forms of cancer, and so such inflammation-induced expression of granulin may promote cancer stem cell activity in this context. Accordingly, the methods or uses of the invention may be of great benefit in the treatment of cancer associated with inflammation, since they are able to inhibit the tendency that may otherwise exist for granulin expression associated with the inflammation to promote cancer stem cell activity.

The inventors have shown that granulin signalling appears to contribute to cancer stem cell activity in a number of different forms of cancer, as illustrated in the Experimental Results section below. Accordingly, the methods or uses of the invention may be of particularly utility in the prevention and/or treatment of a cancer is selected from the group consisting of: breast cancer; prostate cancer; and melanoma.

Of particular interest is the application of the methods and uses of the invention for use in the prevention and/or treatment of breast cancer.

There are a number of different ways in which breast cancers can be classified. One that is of particular relevance in the context of the present invention is with reference to the expression of the oestrogen receptor by cancer cells. The experimental data that the inventors have generated indicate that the methods and uses of the invention will be of particular use in the prevention and/or treatment of cancer associated with oestrogen receptor-positive (ER+) cells. Such cancers represent the most prevalent form of breast cancer.

Furthermore, the inventors' finding that granulin signalling contributing to cancer stem cell activity is mediated via sortilin provides an additional marker by which cancers that are likely to benefit from treatment with agents that inhibit granulin signalling may be identified. Accordingly, the invention provides a method of selecting a regimen for the treatment of a cancer of interest, the method comprising assaying a sample representative of gene expression in the cancer of interest for the presence of a target molecule indicative of the expression of sortilin; wherein

if a target molecule indicative of the expression of sortilin is present in the sample representative of gene expression in the cancer of interest, then a treatment regimen utilising an agent that inhibits granulin signalling should be selected; and
if a target molecule indicative of the expression of sortilin is not present in the sample representative of gene expression in the cancer of interest, then a treatment regimen utilising an agent that inhibits granulin signalling should not be selected.

The invention also provides an additional step in methods of treatment using either agents that inhibit granulin signalling or sortilin activity for the treatment of cancer, the additional step comprising assaying a sample representative of gene expression in the cancer to confirm the presence of a target molecule indicative of the expression of sortilin in advance of treatment.

In the embodiments described above, a sample representative of gene expression in a cancer may be a sample comprising nucleic acids or proteins that are representative of gene expression. A target molecule indicative of expression of sortilin may comprise a nucleic acid encoding sortilin, such as mRNA, or sortilin protein. Suitable means by which such target molecules may be detected include labelled nucleic acid probes or labelled antibodies.

### Treatment Regimens Using the Methods or Medical Uses of the Invention

Medical uses or methods of treatment in accordance with the invention may be used in monotherapies (in which the agent that inhibits granulin signalling is the sole agent providing substantial therapeutic activity).

That said, it may generally be preferred that the methods or medical uses of the invention be utilised in combination therapies, in which the agent that inhibits granulin signalling is used in combination with other anti-cancer therapies, consistent with their use as adjunct therapies suggested elsewhere in the specification.

Suitably, an agent that inhibits granulin signalling is used in combination with a further therapeutic agent. In particular, the agent that inhibits granulin signalling may be used in combination with a further anti-cancer agent.

In the context of the present invention, references to agents of the invention for “use in combination with” or for “treatment in combination with” further anti-cancer agents (such as those that induce tumour hypoxia, as considered below) may be taken as encompassing any effective treatment regimen in which both the agent of the invention and a further anti-cancer agent are provided to a subject in need of cancer treatment.

As discussed further above, the methods and medical uses of the invention may be of particular utility in contexts in which cancer stem cells are found in hypoxic conditions, and such hypoxic conditions may arise as a result of cancer treatment. Accordingly, in a suitable embodiment, the methods or medical uses of the invention may employ an agent that inhibits granulin signalling for use in combination with a further anti-cancer agent that induces tumour hypoxia.

A suitable anti-cancer agent that induces tumour hypoxia may be an inhibitor of new blood vessel formation. Suitable such an agent may be an inhibitor of angiogenesis. Examples of angiogenesis inhibitors able to induce hypoxia that may be used in combination with the agents of the invention include those selected from the group consisting of: bevacizumab, itraconazole, carboxyamidotriazole, suramin, thrombospondin, tecogalan, and marimastat.

As referred to above, the invention also relates, in its seventh aspect, to cell culture products that contain granulin, or its precursors or biologically active cleavage products. The cell culture product may be a cell culture medium that contains the granulin. Alternatively it may be a supplement for use in a cell culture medium thereby allowing a requisite amount of granulin to be added to any cell culture medium.

In an eighth aspect the invention provides a method of promoting survival in cultured stem cells, the method comprising providing the cultured stem cells with a source of granulin, or a biologically active precursor or fragment of granulin. The source of granulin may be a cell culture product in accordance with the seventh aspect of the invention, as discussed above. In this aspect of the invention the cells being cultured are provided with an amount of granulin sufficient to promote their survival, which may readily be determined with reference to the cell type, cell density, or other similar parameters.

The invention will now be described further with reference to the following Experimental Results, and the accompanying Figures in which:

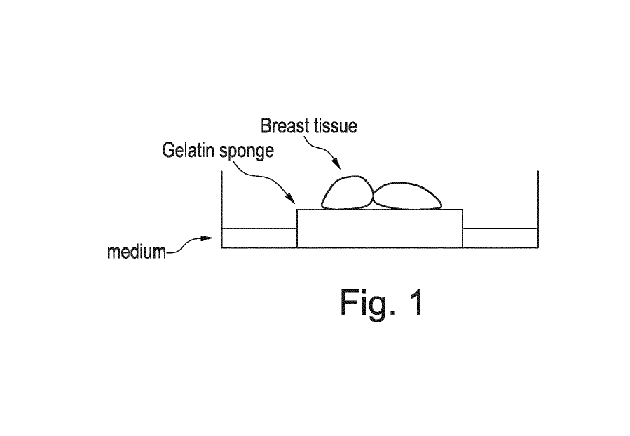

FIG. 1 shows a schematic representation of the ex vivo explant model for culturing human breast tissue

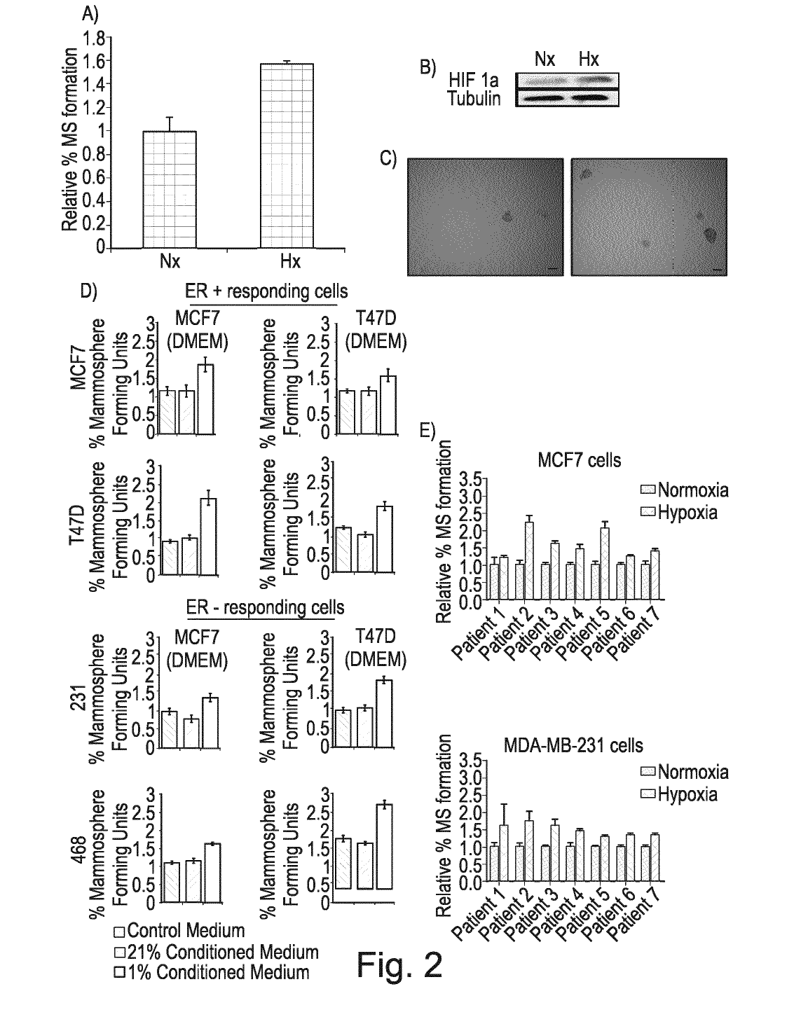

FIG. 2 demonstrates that hypoxia directly and indirectly influence BCSC-like populations in ER positive breast cancer. Culture of ER positive MCF7 cells in hypoxic conditions for 48 hours increases mammosphere formation relative to cells cultured in normoxia (A). Hypoxic conditions were confirmed by western blot analysis of HIF1α expression (B). Light microscope images record the increase in MS formation observed at a 5× magnification view. Scale bar shows 200 μm diameter (C). Treatment of ER positive cell lines (MCF7 and T47D) or ER negative cell lines (MDA-MB-231, MDA-MB-468) with conditioned media (CM) from hypoxic cultures of ER positive cell lines (MCF7 and T47D) increase their mammosphere forming capacity (D). CM from Patient ex vivo explants regulates MS formation of MCF7 or MDA-MB-231 cell lines (E).

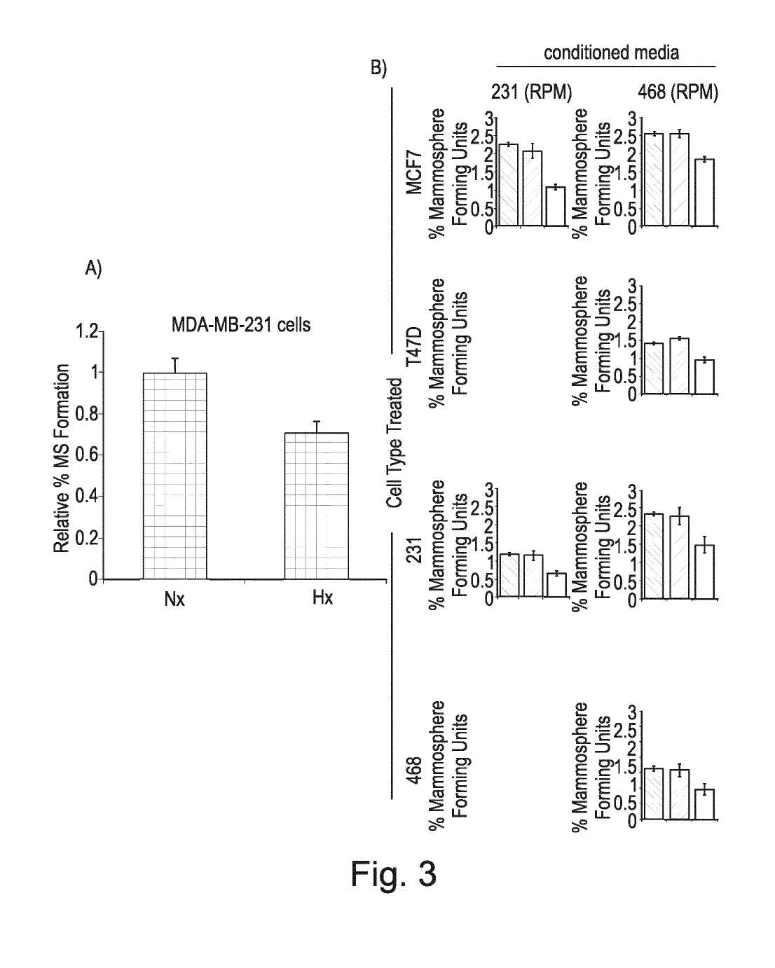

FIG. 3 illustrates that hypoxia negatively regulates CSC propogation in ER negative BC. Culture of ER negative MDA-MB-231 cells in hypoxic conditions for 48 hours; decreases their mammosphere formation capacity (A). Treatment of ER positive cell lines (MCF7 and T47D) or ER negative cell lines (MDA-MB-231, MDA-MB-468) with conditioned media (CM) from hypoxic cultures of ER negative cell lines (MDA-MB-231, MDA-MB-468) decrease their mammosphere forming capacity (B).

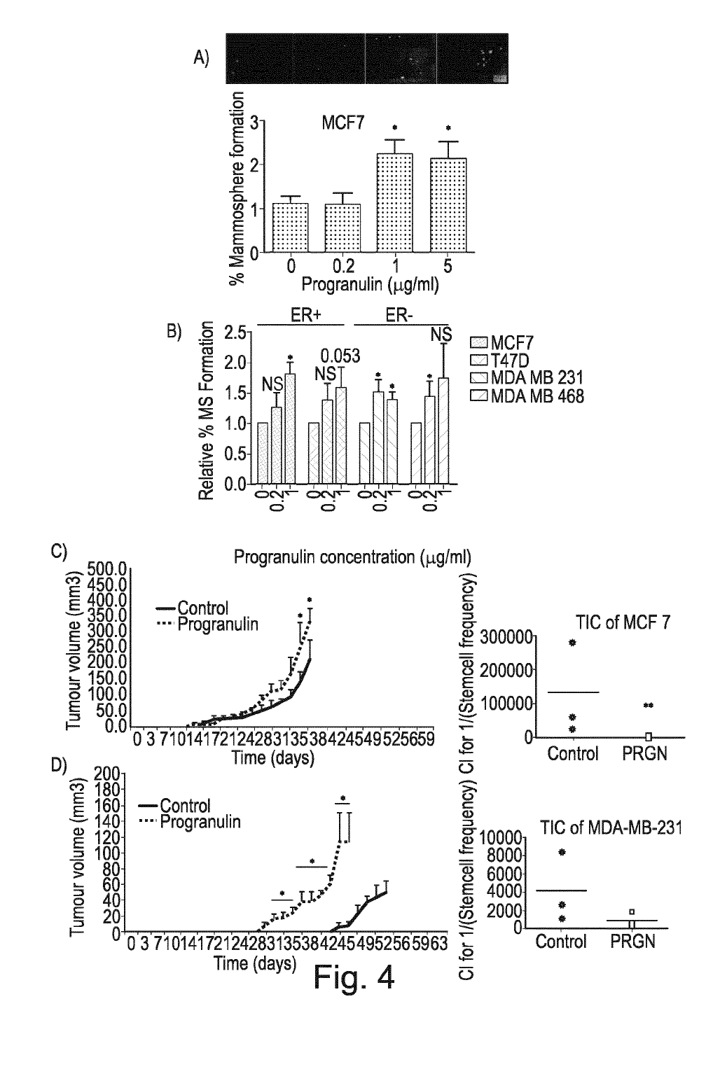

FIG. 4 illustrates that progranulin treatment increases BCSC populations of breast cancer cells. Using cytokine array technology, a list of peptides was identified that become secreted into CM by ER positive cells MCF7 and T47D in response to hypoxia of which PRGN was selected as the most effective at inducing MS formation in both ER positive and ER negative cell lines (graph represents mean relative % MS formation, error bars represent St dev of three independent experiments n=3) (A & B). MCF7 (C) and MDA-MB-231 (D) cells pre-treated with PRGN for 48 hrs required a lower number of cells to initiate a tumour in vivo compared to control cells. Graph representing mean tumour volume of both PRGN treated cells and control cell during the experimental timeframe. Whisker box plot shows the difference in tumour initiating frequency between the two groups.

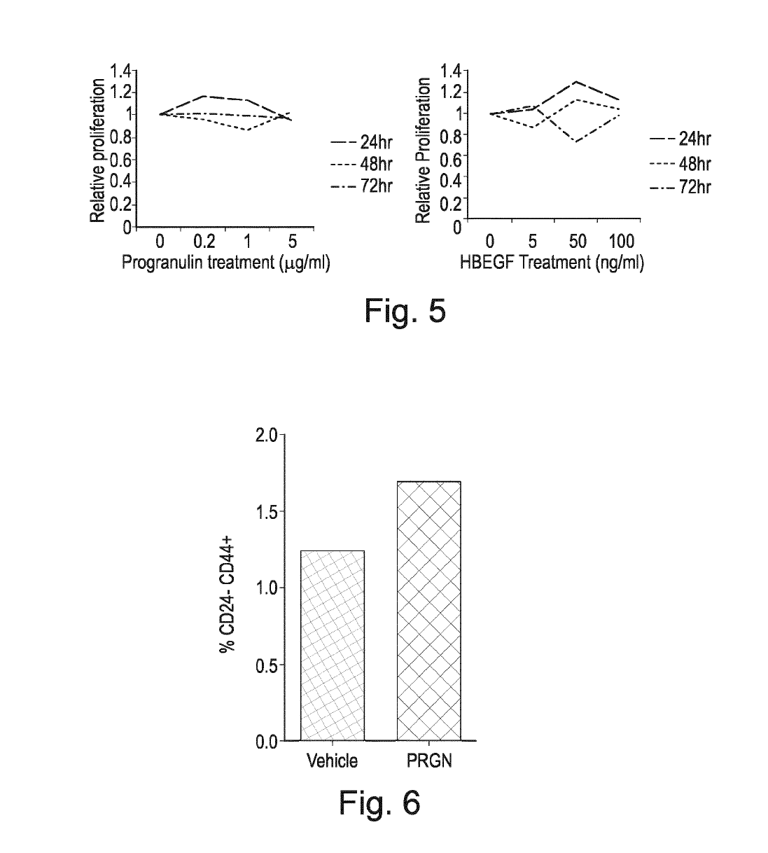

FIG. 5 demonstrates that treatment with Progranulin or HB-EGF does not affect proliferation of MCF7 cells.

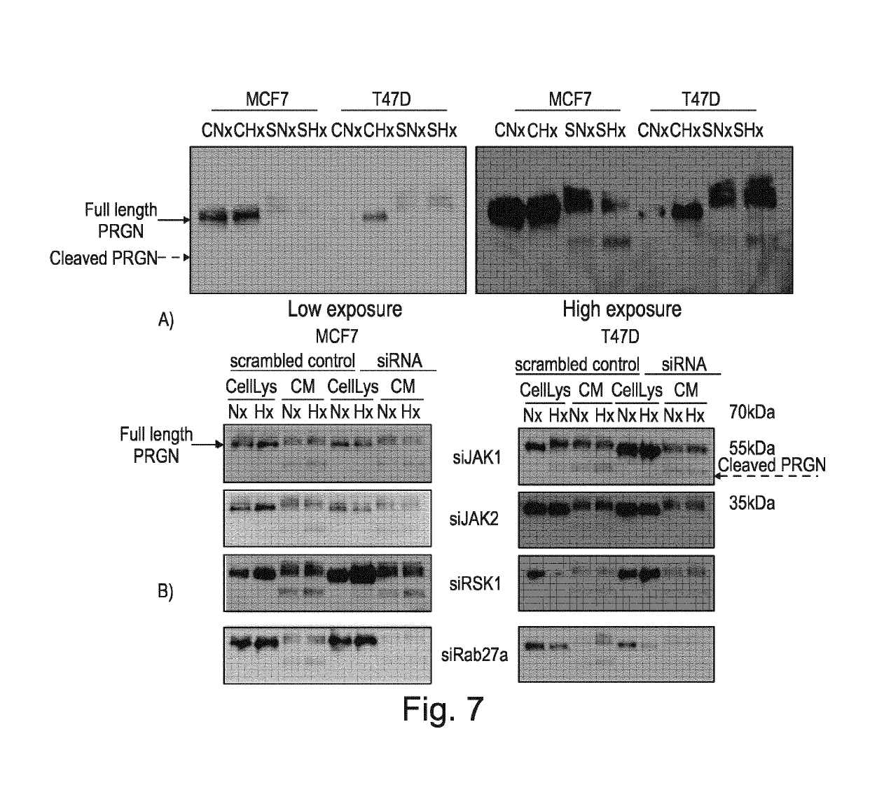

FIG. 6 shows treatment with Progranulin increases the CD24− CD44+ cell population in MCF7 cells.

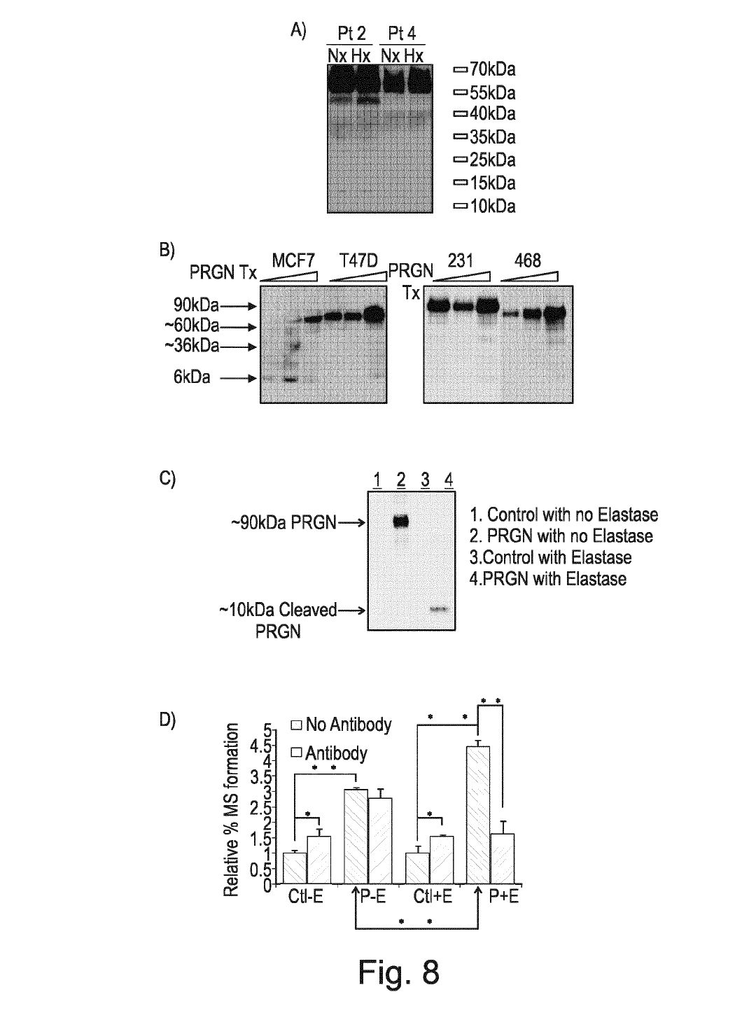

FIG. 7 shows progranulin cellular expression and external cleavage increases in response to hypoxia. Analysis of PRGN levels in ER positive (MCF7 and T47D) cell lysates reveal in increase in full length PRGN expression in response to hypoxia (CHx) when compared to normoxia (CNx), cell line CM reveals very little difference in full length PRGN; however a large variation is evident in cleaved PRGN in response to hypoxia (SHx) (A). Both cell lysate protein expression and externally cleaved PRGN expression is dependent on JAK1 and JAK2 and not RSK1. Secretion of fragmented PRGN but not internal hypoxia induced expression is dependent on Rab27 alpha (B).

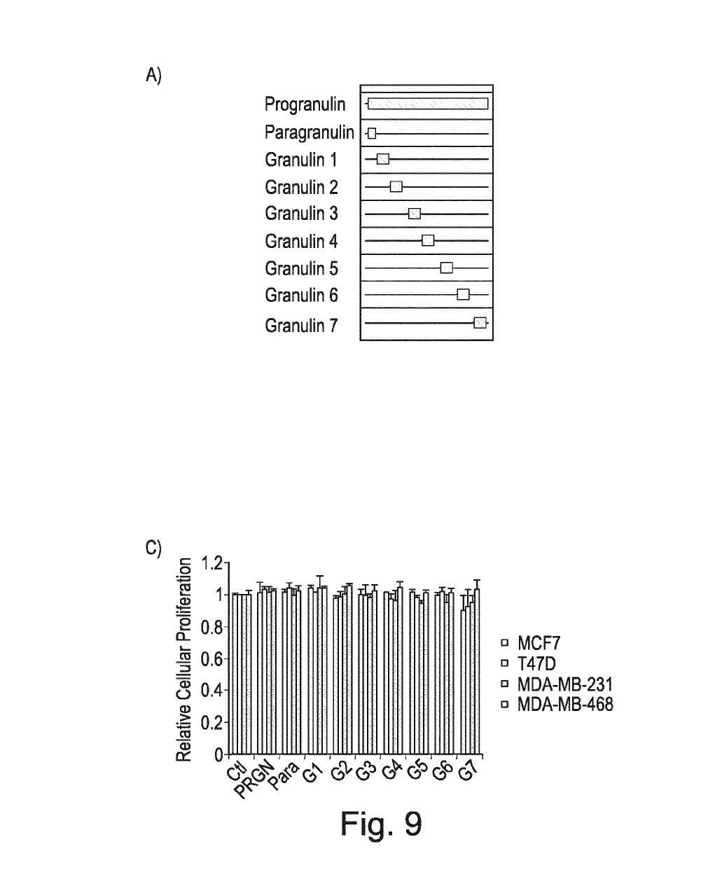

FIG. 8 illustrates that progranulin is internalized and active in both full length and cleaved forms. Conditioned media from patient ex vivo explants show very little difference in full length PRGN; however a large variation is evident in cleaved PRGN in response to hypoxia (SHx) (A). Western blot analyses of cell lysates indicate internalization of PRGN in both full length and fragmented forms when cells are treated with a concentration gradient of PRGN for 48 h (B). Enzymatically cleaved PRGN (C) was used to treat MCF7 cells for 48 hrs prior to MS assay. This resulted in an increase in MS formation when compared to full length PRGN treated cells. This increase observed in cleaved PRGN was attenuated when cells were treated with an anti-PRGN antibody that recognizes cleaved PRGN (D). Enzymatic cleavage of progranulin was performed by incubating 5 ug/ml of full length progranulin with 0.3 ug/ml of elastase enzyme in a TRIS-HCl buffer at 37° C. for 18 hrs. Enzyme activity was inhibited with 0.5% formic acid, and successful cleavage was confirmed by western blot analysis. Treatment of cells was done with addition of either cleaved or uncleaved (no enzyme control) progranulin to a final concentration of 1 μg/ml for 48 hours prior to MS assay (B).

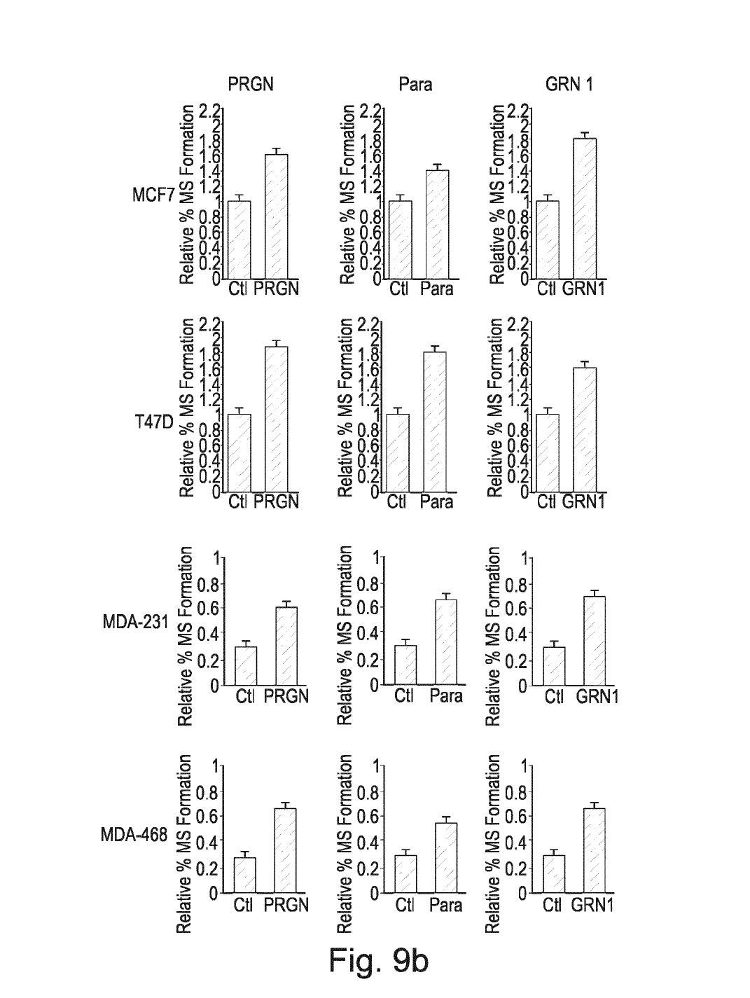

FIG. 9 demonstrates that GRN domains paragranulin, GRN 1 and GRN 5 increase MS formation of breast cancer cell lines without effecting proliferation. GRN domains were constructed by Activotech corp, Cambridge, U.K. (A). Each domain was used to treat MCF7, T47D, MDA-MB-231 and MDA-MB-468 cell lines for 48 hrs (1 ug/ml) prior to MS assay. Domains paragranulin, GRN 1 and GRN5 were observed to increase MS formation (B). All cell lines were also subjected to an Alamar Blue assay to determine the effect of these GRN domains on proliferation over a 48 hr time period. Results indicate no change in cell growth using these conditions (C).

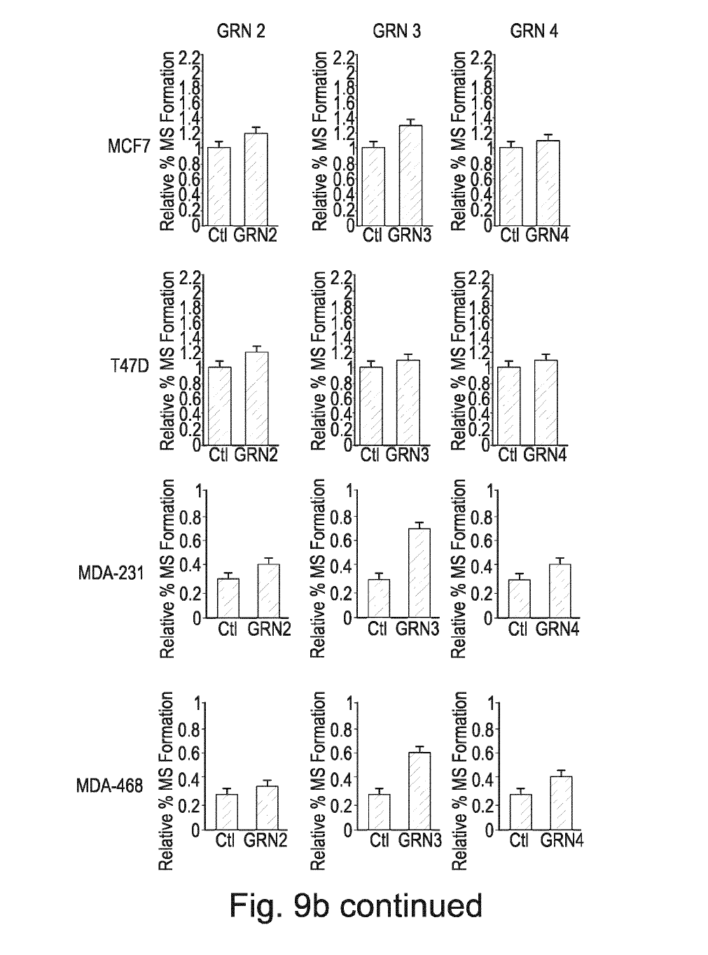

FIG. 10 illustrates that combinations of GRN domains paragranulin, GRN 1 and GRN 5 do not show an increase MS formation compared to full length PRGN in MCF7 (A) and T47D (B) cells. Each treatment of MCF7 and T47D cells was done for 48 hrs using a maximum concentration of 3 ug/ml of peptide/peptides prior to MS assay. Results represent mean relative MS formation ±StDev of two independent experiments (n=2).

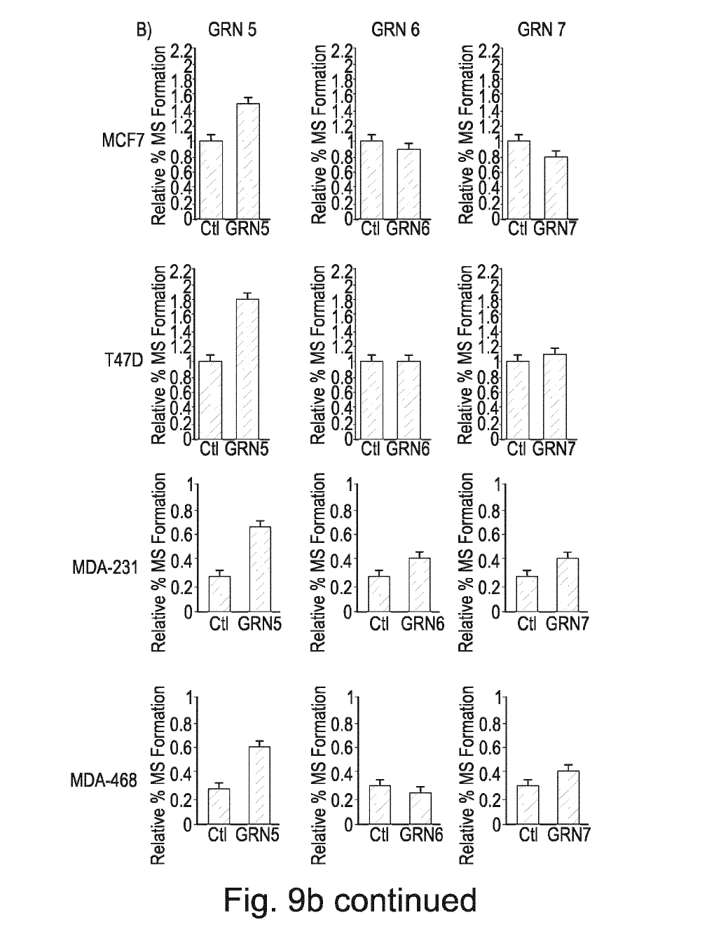

FIG. 11 sets out a number of graphs illustrating that mammosphere formation in response to progranulin treatment is reduced in cancer cell lines in which sortilin expression has been knocked down.

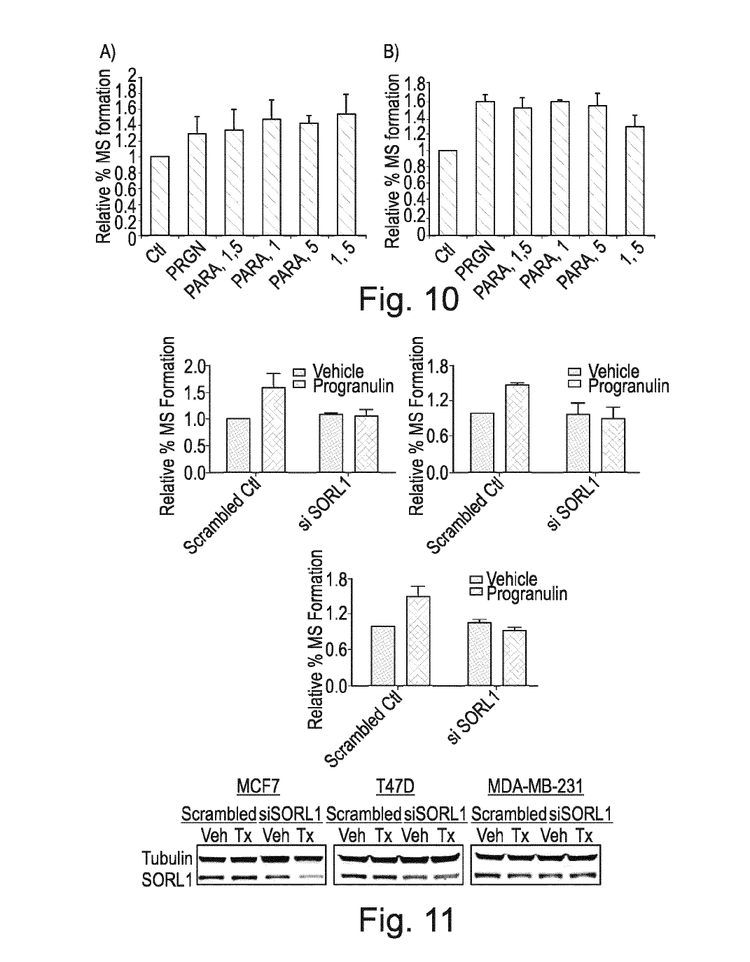

FIG. 12 shows immunohistochemical staining representative of (from left to right) “low”, “medium” and “high” expression of sortilin in clinical samples of breast cancer.

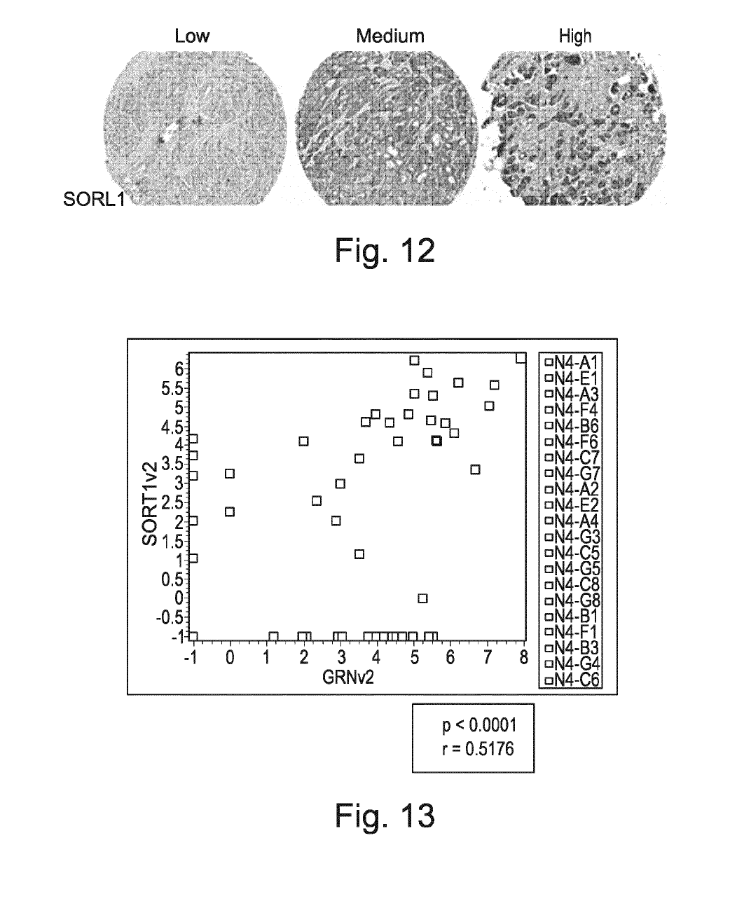

FIG. 13 illustrates the association between progranulin and sortilin expression in MCF7 cells.

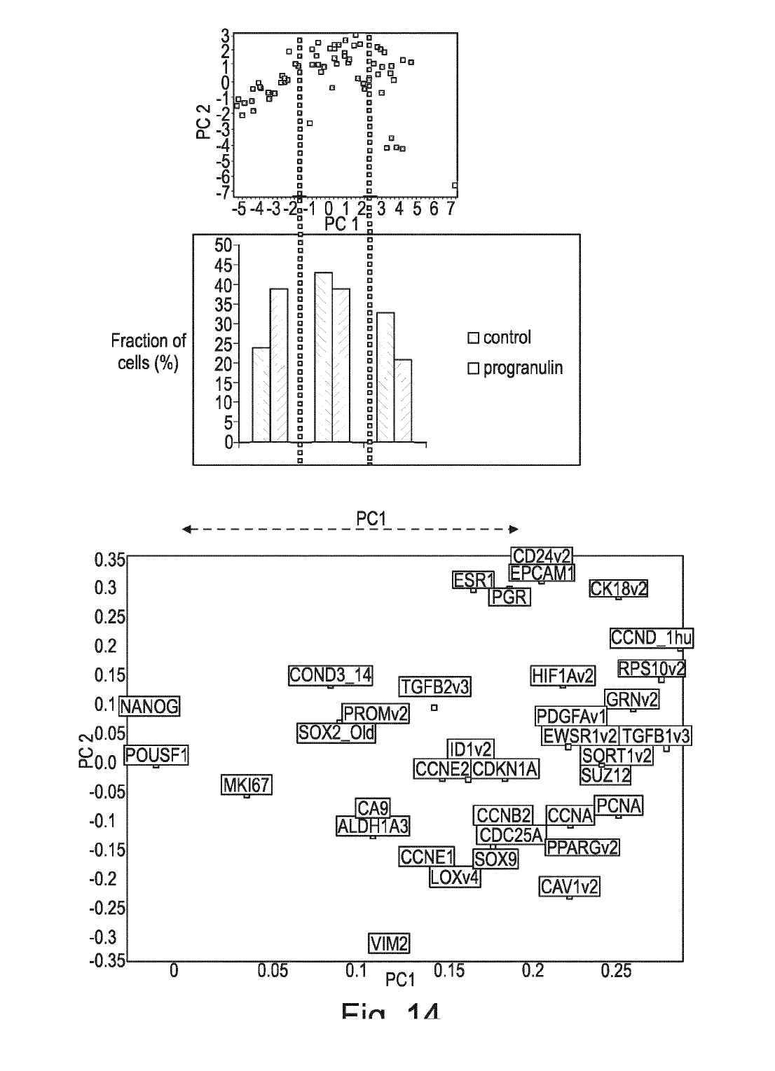

FIG. 14 shows partial component analysis of PCR data from single cells treated with progranulin or control.

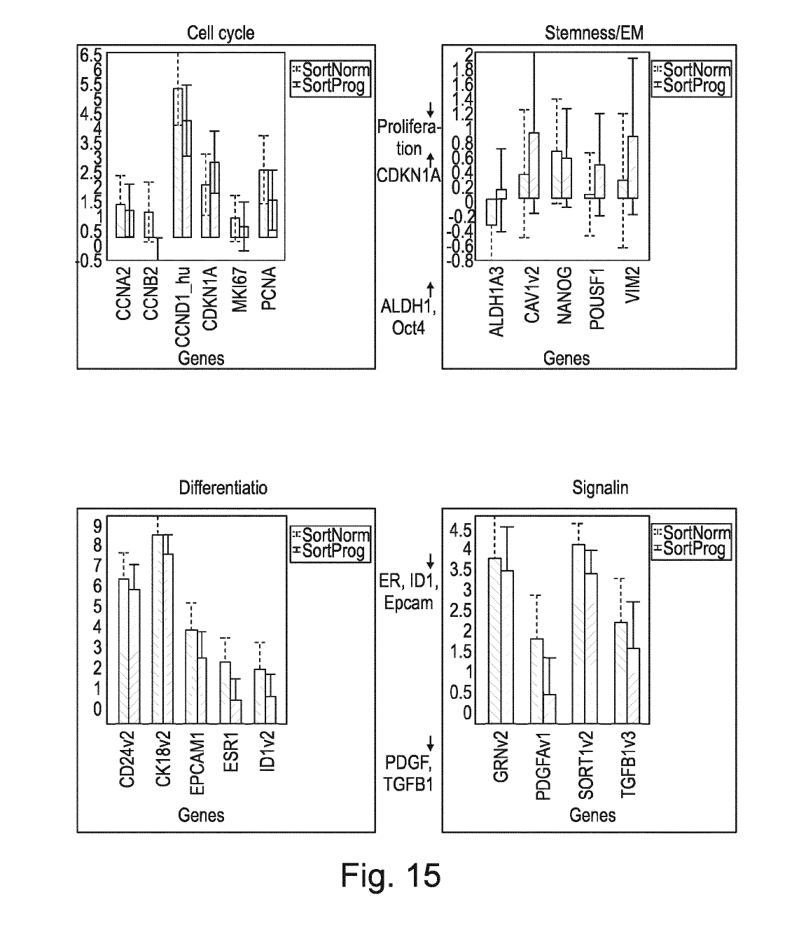

FIG. 15 sets out a number of graphs illustrating the impact of progranulin treatment on expression of a range of different markers by sortilin positive cells.

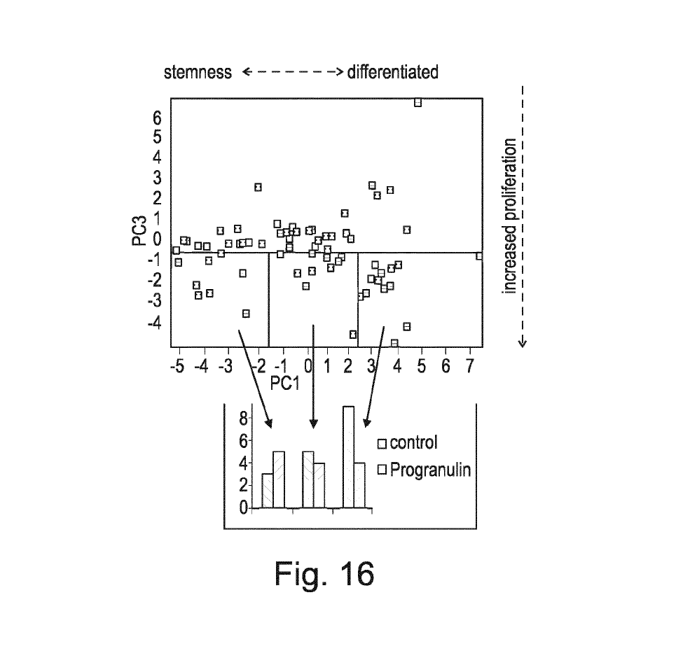

FIG. 16 shows the impact of treatment with either progranulin or control on proliferation of various cell populations sorted with respect to their level of differentiation.

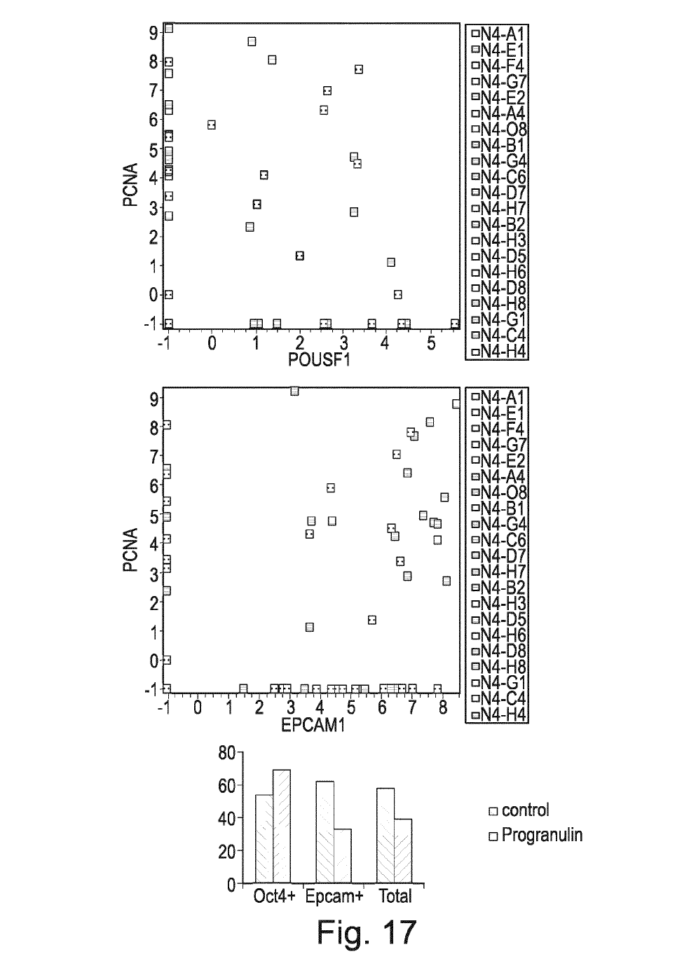

FIG. 17 illustrates that progranulin treatment reduces proliferation in non-stem cells (“total” or expressing the differentiation marker Epcam), while increasing proliferation among stem cells (expressing the marker Oct4).

### EXPERIMENTAL RESULTS

### Study 1—Role of Progranulin in Cancer

### Abstract

The microenvironmental niche of a solid tumour such as breast cancer plays a direct role in the behavior of cancer cells. One of these contributing factors is low oxygen supply, which is common in solid tumours with low blood supply and high, compact cell numbers or can alternatively be induced by hypoxia inducing therapies. This hypoxic stress has been implicated in influencing the stem cell-like populations in breast cancer. Recent studies have uncovered a diverse response of breast cancer stem cell (BCSC)-like populations to hypoxia, based on ER expression. ER positive cell populations respond to hypoxia by increasing their BCSC population, while conversely ER negative cell types show decreased levels of BCSC numbers. Interestingly, we have now observed that this effect not only appears in cells under hypoxic conditions but by transfer of hypoxia treated culture media. This suggests that hypoxia induces secretory elements from epithelial cells that directly influence the BCSC populations of other cells. Protein array analysis of the hypoxic culture media of ER positive cell lines MCF7 and T47D identified progranulin as a significantly upregulated peptide in response to hypoxia. To estimate the contribution of the upregulated peptides on BCSC like behavior, cells were treated with progranulin at increasing concentration producing increased mammosphere formation in both ER positive as well as ER negative breast cancer cell lines. There was not any marked increase of proliferation after progranulin treatment suggesting that the increase of mammosphere formation was caused by a genuine dedifferentiation process with an increased amount of cancer initiating stem cells after treatment. Further supporting this observation was a significantly increased tumour initiating capacity of progranulin treated MCF7 and MDA-MB-231 breast cancer cells in immunocompromised mice. Detailed assessment of progranulin levels in cell line supernatants after hypoxia exposure further revealed an increase of the total amount of progranulin including specifically cleaved progranulin domains. Hypoxia induced cellular expression of PRGN was shown to be dependent on JAK1 and JAK2. Secretion of cleaved PRGN was shown to be dependent on JAK1, JAK2 and Rab27 alpha. Conflicting with current data, RSK1 was not observed to effect PRGN expression or secretion. Enzymatically cleaved progranulin showed a pronounced mammosphere activating ability compared to full length progranulin. This activating function by the cleaved progranulin could be specifically blocked by adding progranulin antibodies to the cells illustrating a strategy that could be used to block unfavorable and stem cell activating effects of secreted progranulin and specifically site dependent cleavage of progranulin. Similar findings were obtained using an elastase inhibitor together with progranulin treatment supporting a key function for cleaved progranulin in mediating the dedifferentiation process. Conditioned media from patient ex vivo explants incubated in both normoxia and hypoxia was used to test the effect on MCF7 cells and MDA-MB-231 cells stem cell proportions. Conditioned media from all patients with ER-positive breast cancer increased mammosphere formation in ER positive and ER negative cell lines following hypoxic incubation. There was also an increase in cleaved PRGN in patient explant CM. This study uncovers a novel mechanism in which breast cancer cells respond and communicate to environmental changes altering the fraction of cancer stem cells in a coordinated manner. This study also identifies cleaved progranulin as a major component, mediating this effect in ER+ breast cancer. Targeting progranulin secretion, cleavage and cellular effects represent an attractive novel treatment approach for breast cancer subtypes endogenously exposed to hypoxia or being treated with hypoxia inducing regimes. This strategy would limit the detrimental cancer stem cell increase in the secretion based enlarged hypoxic niches thereby limiting the progression of the disease and improving patient outcome.

### INTRODUCTION

In this study, ER positive cells where observed to secrete signaling peptides that direct BCSC populations in response to hypoxia. The peptide progranulin was effective at inducing the stem cell-like phenotype in both ER positive and ER negative breast cancer. This protein has previously been attributed to many functional properties in breast cancer, including proliferation and invasion. Elevated expression of this peptide has also been associated with recurrent disease with patients receiving endocrine therapy; however, this is the first study to highlight its role in breast cancer stem cell regulation (see appendix for more information on PRGN). Data from this study may aid in identifying a contributing pathway to stem cell propagation in breast cancer and reveal novel therapeutic strategies to combat both initial and recurrent disease.

### Materials and Methods

### Cell Lines

MCF7, T47D, MDA-MB-231, and MDA-MB-468 were purchased from American Type Culture Collection. Lines were authenticated by multiplex-PCR assay using the AmpF/STR system (Applied Biosystems) and confirmed as mycoplasma free. Monolayers were grown in DMEM (DMEM/10% FCS/2 mmol/L L-glutamine/PenStrep, MCF7, and T47D), or RPMI medium (RPMI/10% FCS/1% sodium pyruvate/2 mmol/L Lglutamine/PenStrep, 231, and 468). Cells were maintained in a humidified incubator at 37° C. at an atmospheric pressure of 5% (v/v) CO2/air.

### Hypoxic Cell Culture

Cells we incubated for 48 hours in the SCI-tiveN hypoxic workstation (Ruskinn) in 1% 02, 5% CO2, and 94% N2 in a humidified environment at 37° C. Cells were plated, cultured, and harvested within the workstation to maintain hypoxia at all times. Confirmation of hypoxic conditions was carried out using Western blotting to measure expression of HIF-1α.

### Peptide Treatments

Peptide mimetics CXCR2 (Abcam ab24230), HB-EGF (R&D Systems 259-HE-050) and PRGN (Nordic Biosite AG-40A-0068-C050) were reconstituted in sterile PBS upon delivery, aliquoted and stored at −20° C. Working stock concentrations were achieved by dilution in culture media. Cell lines were treated with indicated concentrations of peptide for 48 hours at 37° C. 5% CO₂ and 21% O₂.

### Patient Ex Vivo Explants

Breast cancer tissue was obtained with written informed consent through the Sahlgrenska Cancer centre. An 8 mm core of tissue was dissected into 1 mm3 pieces and cultured in duplicate on a pre-soaked gelatin sponge (Johnson and Johnson, New Research) in 6-well plates containing 2 ml DMEM with 10% FCS and 1% penicillin streptomycin solution (FIG. 1) (Centenera et al, 2012; Dean et al, 2012). Tissues were cultured in either normoxic (21% oxygen) or hypoxic (1% oxygen) conditions at 37° C. for 48 h then formalin-fixed and paraffin embedded or preserved in RNAlater (Invitrogen, San Diego, Calif.). Conditioned culture media was taken from explants and centrifuged at 1300 rpm for 5 mins to remove cellular debris. This conditioned media was then used to either treat cell lines or undergo Western blot analysis. Tumour cell histology was obtained following completion of experiments in order to avoid analytical bias.

### Mammosphere Culture

Mammosphere culture was carried out as described by Shaw et al, 2012, and spheres were counted on day 5 to avoid counting of any mammospheres that may have arisen from normal epithelial cells.

### Western Blotting

25 μg of protein was separated on an SDS-PAGE and transferred to Hybond-C Extra nitrocellulose membrane or PVDF membrane. For PRGN detection non-reducing conditions were used as these conditions were optimal for the primary antibody. Primary antibodies included: Actin (Santa Cruz, sc-1616), PRGN (R&D systems) and HIF-1a (610959, BD Biosciences).

### In Vivo Tumor Formation

Cells were injected subcutaneously into NOD scid gamma mice. Ninety-day slow release estrogen pellets (0.72 mg) were implanted subcutaneously 2 days before injection (MCF7 only, Innovative Research of America). MDA-MB-231 cells or MCF7 cells were suspended in a 1:1 mixture of matrigel and mammocult media in serial dilution format. Upon collection, xenograft tumours were divided for both formalin fixation and immunohistochemistry or stored in RNase later for expression analysis.

### Transient Transfections

MCF7 and T47D cells were transfected with 10 uM of siJAK 1, siJAK2 siRSK-2 or siRab27 alpha. Briefly, cells were seeded in T25s in media with no PenStrep added. Cells were incubated at 37° C. overnight. For each mix 500 ul OptiMEM with 10 ul Lipo2000 (Sol A), then mix another 500 ul OptiMEM with 10 ul siRNA (Sol B). Incubate at RT for 5 mins. Mix both Sol A and Sol B together (transfection solution) and incubate for 20 mins at RT. Wash cells in PBS and add transfection solution to cells. Incubate for 4 hours at 37° C. Add 4 ml of normal media to each T25 (2 ml for 6 well plates). Cells were incubated for 48 hours prior in either normoxic (21% O2) or hypoxic (1% CO2) conditions.

### Results:

The hypoxic microenvironment and breast cancer stem cell like behaviour Breast cancer cell lines have been shown to have differential responses to a hypoxic microenvironment wholly dependent on histological status. With ER positive cell lines such as MCF7, hypoxia stimulates an increase in the mammosphere forming capacity of the cell line (FIG. 2A), whereas in the MDA-MB-231 cell line a decrease is observed (FIG. 3A). This observation further implores an investigation of the relationship between hypoxic tumour microenvironments and recurrence in subtypes of breast cancer. As hypoxia is a confined event relative to the entire tumour area, it is of interest to determine how the cells in this confined hypoxic area may influence the entire tumour population. In order to determine the influence hypoxia exposed cells may have on surrounding cell populations; media from hypoxia exposed cells was used to treat breast cancer cell lines under normoxic conditions. Hypoxic media from ER positive cell lines increased mammosphere formation of all breast cancer cell lines used (FIG. 2D). This data suggest that conditioned media from hypoxia exposed cells effect mammosphere formation similar to physical hypoxic conditions. Supporting this data, hypoxic media from ER negative cell lines reduced mammosphere formation in all breast cancer cell lines (FIG. 3B). Using a patient ex vivo explant model, it was observed that hypoxic ER positive breast cancer conditioned media influence breast cancer cell lines in a similar way that is observed with ER positive cell lines MCF7 and T47D (FIG. 2E).

### Identification of Regulatory Elements Responsible for the ER Positive Hypoxic Influence on BCSC Like Propagation

Conditioned media from the ER positive cell lines MCF7 and T47D was analysed for expression changes of a broad range of cytokines in response to hypoxia using cytokine array technology. From this analysis, several peptides were identified to be significantly elevated and one downregulated in both cell lines in response to hypoxic stimulation (data not shown). Peptide mimetics were obtained from various sources and MCF7 cells were treated using a concentration gradient of each upregulated peptide memetic for 48 hours in order to mimic the conditioned media treatment. Analysis of the BCSC-like population was performed by subjecting treated cells to a mammosphere assay. Results suggest that progranulin was most effective at inducing mammosphere formation of MCF7 cells (FIG. 4A). This experiment was then repeated on a panel of breast cancer cell lines. Progranulin successfully increased the mammosphere forming capacity of both ER positive and ER negative cell lines, similiarly to hypoxic conditioned media from ER positive cell lines in previous experiments without notably effecting proliferation (FIG. 4B, FIG. 5). These findings were validated using an in vivo tumour limiting dilution assay and flow cytometry analysis of CD24 and CD44 expression (FIG. 6). In the xenograft study both MCF7 and MDA-MB-231 cells were treated or not treated with progranulin for 48 hours before implantation. This pretreatment resulted in an increased tumour take (MCF7 p<0.001, MDA-MB-231 p=0.06) indicating a higher number of tumour initiating cells in the bulk population of progranulin treated cells (FIG. 4C, 4D).

### Progranulin Expression and Cleavage in Luminal Breast Cancer

In order to validate the findings of the cytokine array, CM from MCF7 and T47D cell lines was analysed for PRGN by western blot. Results of this analysis indicate that hypoxia successfully increased the expression of full length progranulin in cell lysates, but more interestingly the differences in full length PRGN between normoxic and hypoxic conditioned media samples were minimal; however there is a large increase in fragmented PRGN in response to hypoxia (FIG. 7A). These observations were reproduced in some ex vivo explants (FIG. 8A). Frampton et al 2012 describe a mechanism for progranulin regulation in cholangiocarcinoma, suggesting an IL-6-ERK-RSK-1 mediated pathway. Therefore to test the hypothesis that progranulin may be regulated in a non-direct manner via cytokine signaling, a transient knockdown of JAK 1 and JAK 2 were performed as these are essential signaling proteins for cytokine receptors. This resulted in a blockage of hypoxia induced PRGN expression and secretion of cleaved PRGN (FIG. 7B). Conversely to Framton et al, 2012 findings RSK-1 was not involved in the regulation of hypoxia mediated PRGN expression or secretion. The exosome regulator Rab27 alpha was observed to be a mediator of PRGN secretion but not hypoxia induced expression (FIG. 7B). This may also be a valuable target for inhibiting hypoxic induced secretions from cancer cells. When cells are treated with full length PRGN for 48 hours, western blot analysis reveals internalization of both full length and fragmented forms of progranulin suggesting cleavage of PRGN occurs in responding cells as well as progranulin producing cells (FIG. 8B). To assess the functional relevance of progranulin cleavage, full length progranulin was incubated with or without elastase enzyme for 18 hours. The product of this was successfully cleaved progranulin (FIG. 8C), which was used to treat cell for 48 hours prior to assessment of mammosphere forming capacity. These data suggest that cleaved progranulin is more effective at increasing mammosphere forming capacity than the full length form. Treatment of cells with anti-PRGN antibody inhibits the effect of cleaved PRGN on mammosphere formation, but not full length PRGN (FIG. 8D).

Progranulin is known to be enzymatically cleaved into 8 different domains (FIG. 9A). These domains were constructed (Activotech) and used to treat breast cancer cell lines (1 ug/ml) for 48 hours. Cells were then subjected to mammosphere assay. Results suggest PRGN domains paragranulin, GRN1 and GRN 5 are effective at inducing MS formation in both ER positive and ER negative cell lines (FIG. 9B). GRN domains had no significant effect on proliferation of any cell type (FIG. 9C). To determine if there is an additive effect when combining these peptides, MCF7 and T47D cells were treated with different combinations of paragranulin, GRN 1 and GRN5 and compared to cells treated with progranulin at the identical protein concentration of each combined treatment (3 ug/ml) using a mammosphere assay. No significant difference was observed between the combination of these GRN domains and progranulin in terms of mammosphere formation (FIG. 10).

### Study 2—Role of Sortilin in Progranulin Signalling Associated with Cancer

### 2.1 Sensitivity of Cancer Cell Lines to Progranulin is Reduced on Knockdown of Sortilin

The results reported in Study 1 (above) had established that treatment of cancer cells from the ER positive cell lines MCF7 and T47D with progranulin induced mammosphere formation characteristic of breast cancer stem cell like behaviour.

The inventors used siRNA to knock down SORL1 (the gene encoding sortilin) for 48 hours in ER positive cell lines MCF7 and T47D, and in the ER negative cell line MDA-MB-231. The cells were then treated or untreated with progranulin at a concentration of 1 μg/ml for a period of 48 hours, and assessed for their potential to form mammospheres. The results of this study are set out in FIG. 11. Here it can be seen that, in each of the cell lines tested, knock down of sortilin prior to progranulin treatment reduced cell sensitivity to progranulin, and thus reduced mammosphere formation indicative of cancer stem cell like behaviour.

### 2.2 Association of Sortilin Expression and Progranulin Expression in Breast Cancer Samples

The association between sortilin and progranulin was investigated (using immunohistochemstry to indicate expression) in a tissue microarray containing clinical material from 130 primary breast cancer samples. SORL1 expression was found to be significantly associated with PRGN expression. Illustrative images of immunlabelling of sortilin expression in these samples are shown in FIG. 12.

Expression of sortilin and progranulin was also compared with expression of hypoxia induced factor 1 (HIF1a). In the ER positive samples SORL1 was not associated with hypoxia. This finding indicates that cells in non-hypoxic zones may be induced to breast cancer stem cell-like activity if progranulin is present, and thus suggests that inhibition of progranulin signalling may be of therapeutic utility in all areas of ER positive cancers, not just hypoxic regions.

In contrast when expression of sortilin and HIF1a was compared in the ER negative population, sortilin was found to be associated with hypoxia. This finding suggests that in the case of ER negative cancers, the activity of progranulin in hypoxic areas may cause breast cancer stem cell like activity, and that in such cancers inhibition of granulin signalling is likely to be of greatest clinical benefit in hypoxic zones.

The clinical associations between progranulin, sortilin, HIF1a and CA-9 in ER positive and ER negative cancers can be summarised as follows:

ER+ population
Progranulin is associated with HIF1a and CA-9
Progranulin is associated with sortilin
Sortilin is not associated with HIF1a or CA-9
ER− population
Progranulin is not associated with HIF1a or CA-9
Progranulin is associated with sortilin
Sortilin is associated with HIF1a and CA-9

Investigation of progranulin and sortilin expression at the single cell level in MCF7 cells confirmed the association of these molecules, and also a significant lint with CCND1 and PCNA (both markers of cell proliferation), CK18 and Epcam (both markers of differentiation/non-stemness) and PDGF.

The association between progranulin and sortilin at the single cell level is graphically illustrated in FIG. 13.

### 2.3 Partial Component Analyses (PCA) of Single Cell PCR Data

PCR was used to investigate the response to progranulin of MCF7 cells that were either positive or negative for sortilin expression (33 cells for each). The data generated (illustrate in FIG. 14) showed that cells expressing sortilin respond to treatment with progranulin (2 days) by induction of a stem cell phenotype that shows reduced differentiation. However, this response was not observed in cells where sortilin expression was absent.

This finding further illustrates the utility of determining the sortilin status of cancer cells when considering whether or not they are likely to gain therapeutic benefit from treatment with agents that inhibit granulin signalling, or that inhibit sortilin activity. If sortilin is expressed, then treatment with agents of the sort set out above may be of therapeutic utility. If cancer cells do not express sortilin, then there is likely to be little therapeutic benefit from treatment with agents that inhibit granulin signalling, or that inhibit sortilin activity.

2.4 Effect on Gene Expression of Treatment of Sortilin Positive Cancer Cells with Progranulin

The inventors investigated the impact of progranulin treatment on individual sortilin positive cells. The results, set out in FIG. 15, illustrate that when compared to untreated cells, progranulin treatment:
Generally reduced expression of markers of proliferation
Increased expression of cyclin dependent kinase inhibitor CDKN1A
Increased expression of markers of stemness such as ALDH1 and Oct4
Decreased expression of markers of differentiation such as ER, ID1 and Epcam
Decreased expression of markers associated with signalling such as PDGF and TGF-β1

### 2.5 Effect of Progranulin Treatment on Proliferation of Different Populations of Sortilin Positive Cells

The inventors investigated the effect of progranulin treatment on proliferation of a number of different populations of sortilin positive cells. The results of these studies are set out in FIGS. 16 and 17.

In FIG. 16 it can be seen that treatment with progranulin decreases proliferation in the general population of cells, and causes a more marked decrease in proliferation among the pool of differentiated cells. In contrast, progranulin treatment slightly increases proliferation among stem cells.

In FIG. 17 it can be seen that progranulin treatment reduces proliferation in sortilin positive cells generally, and specifically reduces proliferation among the differentiated (Epcam+ pool of cells). Again, progranulin treatment is shown to increase proliferation slightly among stem cells (positive for Oct4).

1.-44. (canceled)

45. A method for treating cancer in a subject, the method comprising administering to the subject an agent that inhibits sortilin activity.

46. The method of claim 45, wherein cancer stem cell activity is reduced in the subject.

47. The method of claim 45, wherein the cancer is selected from the group consisting of: breast cancer; prostate cancer; and melanoma.

48. The method of claim 45, wherein the cancer is breast cancer.

49. The method of claim 48, wherein the cancer comprises oestrogen receptor positive (ER+) cells.

50. The method of claim 49, wherein the cancer is luminal A breast cancer

51. The method of claim 45, wherein the cancer is associated with a solid tumour.

52. The method of claim 45, wherein the cancer is associated with a hypoxic tumour.

53. The method of claim 45, wherein the agent is an agent that reduces sortilin expression or a function-blocking anti-sortilin antibody.

54. The method of claim 53, wherein the agent is an agent that reduces sortilin expression selected from the group consisting of: siRNA, RNAi and antisense oligonucleotides.

55. The method of claim 45 which is an adjuvant therapy.

56. The method of claim 55 which is for use after surgery.

57. The method of claim 45, wherein the method is for the treatment of metastatic cancer or for the prevention of metastases.

58. The method of claim 45, wherein the agent is administered in combination with a further therapeutic agent.

59. The method of claim 58, wherein the further therapeutic agent is a further anti-cancer agent.

60. A method according to claim 59, wherein the further anti-cancer agent is an agent that induces tumour hypoxia.

61. The method of claim 60, wherein the further anti-cancer agent is an inhibitor of angiogenesis.

62. The method of claim 61, wherein the further anti-cancer agent is selected from the group consisting of: bevacizumab, itraconazole, carboxyamidotriazole, suramin, thrombospondin, tecogalan, and marimastat.

63. The method of claim 59, wherein the agent is administered in combination with radiotherapy.

64. A method of reducing cancer stem cell activity in a subject, the method comprising providing to the subject in need of such a reduction in cancer stem cell activity an amount of an agent that inhibits sortilin activity sufficient to reduce cancer stem cell activity.

65. A method of screening an agent for suitability for use in methods of reducing cancer stem cell activity, medical use to reduce cancer stem cell activity in the treatment of cancer, and medical use in the treatment of cancer, the method comprising assessing the ability of the agent to inhibit sortilin activity.
